(19) United States
(12) Patent Application Publication
(10) Pub. No.: US 2024/0285679 A1
(43) Pub. Date: Aug. 29, 2024

(54) COMPOSITIONS AND METHODS FOR VISION IMPROVEMENT

(71) Applicant: Azura Ophthalmics Ltd., Tel Aviv (IL)

(72) Inventors: Charles BOSWORTH, Las Vegas, NV (US); Yair ALSTER, Tel Aviv (IL); Hila EPSTEIN-BARASH, Shoham (IL); Omer RAFAELI, Udim (IL); Marc GLEESON, Longueville (AU)

(21) Appl. No.: 18/573,953

(22) PCT Filed: Jun. 22, 2022
(86) PCT No.: PCT/IB2022/000343

§ 371 Date: Dec. 22, 2023

Related U.S. Application Data
(60) Provisional application No. 63/214,690, filed on Jun. 24, 2021.

(51) Int. Cl. A61K 33/04; A61K 9/00; A61P 27/10

(52) U.S. Cl. CPC A61K 33/04; A61K 9/0048; A61P 27/10

(57) ABSTRACT

Described herein are compositions and methods for the treatment of vision loss and/or dry eye syndrome. Such compositions comprise keratolytic agents, such as salicylic acid, selenium disulfide, or the like. Topical administration of such compositions to the inner surface of the eyelid provides therapeutic benefit to patients suffering from vision loss and/or dry eye syndrome.

### CROSS REFERENCE

This application claims the benefit of U.S. Provisional Patent Application No. 63/214,690, filed on Jun. 24, 2021, which is incorporated by reference in its entirety.

### BACKGROUND OF THE INVENTION

Vision can be adversely effected by inflammation of corneal tissue and other parts of the eye's surface that prevent the tissues from functioning properly. Common causes of inflammation include, for example medications (e.g., antihistamines, beta-blockers, diuretics, etc.), being in a dry or smoky environment, medical conditions (e.g., diabetes, autoimmune diseases such as rheumatoid arthritis, etc.), herpes, and eye surgeries.

Often inflammation of eye tissue results in symptoms indicative of dry eye syndrome. Dry eye syndrome is a common condition with symptoms ranging from poor night vision, a burning, itching, or stinging sensation in the eyes, light sensitivity, watery eyes, red and/or sore eyelids, and mucus secretions from the eye. A primary cause of dry eye syndrome is a dysfunction of one or more of the oil glands and/or tear ducts of an individual which lead to inflammation of corneal tissue.

Current treatments for dry eye syndrome include treatments to increase, maintain or trigger tear production and heal inflammation.

### SUMMARY OF THE INVENTION

Disclosed herein is a composition comprising a keratolytic agent. In some embodiments the composition provided herein is used in a method of treating vision loss by ophthalmic administration of the composition to an ocular surface or surrounding tissue of the subject, such as the eyelid margin and/or palebra conjunctiva.

Disclosed herein is a method of improving vision in an individual in need thereof, the method comprising providing to the individual a composition comprising a keratolytic agent in an ophthalmically or pharmaceutically acceptable vehicle or carrier, the composition being administered to the eyelid or the eye of the individual in a manner suitable to deliver the keratolytic agent to an eyelid margin of the eyelid or ocular surface of the individual.

The composition can be provided to the individual periodically. The composition can be periodically administered to the individual over a time period of at least 2 weeks. The composition can be periodically administered to the individual over a time period of at least 1 month. Periodic administration can comprise administering the composition at least once a week. Periodic administration can comprise administering the composition at least twice a week.

In some embodiments, the individual has not been diagnosed with a meibomian gland dysfunction (MGD) or lid wiper epitheliopathy (LWE).

In some embodiments, the individual has not been diagnosed with MGD.

In some embodiments, the individual has not been diagnosed with LWE.

In some embodiments, the individual has been diagnosed with a meibomian gland disorder (MGD) or lid wiper epitheliopathy (LWE).

In some embodiments, the individual has been diagnosed with MGD.

In some embodiments, the individual has been diagnosed with LWE.

In some embodiments, the individual does not wear a contact lens.

In some embodiments, the individual does wear a contact lens.

A concentration of the keratolytic agent in said composition can be between about 0.01% to about 10% by weight.

The keratolytic agent can be selenium disulfide. The composition can comprise 0.1% by weight selenium disulfide in an ophthalmically acceptable carrier.

The ophthalmically acceptable carrier can comprise at least one ophthalmically acceptable vehicle and at least one ophthalmically acceptable excipient.

The composition can be administered to at least a portion of a palebra conjunctiva of said eye of said individual.

The composition can be a dispersion or suspension. The composition can be hydrophilic. The composition can comprise an oleaginous base. The composition can be homogeneous.

The composition can be administered via an applicator. The applicator can be a finger of said individual.

The vision can be improved by either questioning patients of their visual function, or improvement measured using a subjective measure or improvement measured using an objective measure.

Visual function can be assessed using a questionnaire. The questionnaire can be OSDI (see, e.g. www.supereyecare.com/resources/OSDI.pdf), VAS, Berkeley Dry Eye Flow Chart (DEFC, see e.g., www.researchgate.net/figure/The-Berkeley-Dry-Eye-Flow-Chart-DEFC-https-doiorg-101371-journalpone0190752g001_fig1_322688369) or CLDEQ-8 (see, e.g., cdn-links.lww.com/permalink/icl/a/icl_2016_04_08_hicksoncurran_15-146_sdc1.pdf).

The vision function can be improved by at least two points as measured by an OSDI vision function scale after one month of periodic administration. The vision function can be improved by at least five points as measured by an OSDI vision function scale after two months of periodic administration. The vision function can be improved in aspects including reading, driving at night, working with a computer and watching TV.

The subjective measure can be any single or combination of visual acuity, hyperacuity, contrast sensitivity, glare visual acuity, visual acuity using PAM, visual acuity under photopic conditions and visual acuity under mezopic conditions. The objective measure can be any single or combination of an Visual Quality Aberrometer, Objective Scatter Image, and a Double Pass System. In specific embodiments, the subjective measure is visual acuity.

### BRIEF DESCRIPTION OF THE DRAWINGS

FIG. 1 illustrates a cross-sectional schematic of an exemplary normally functioning eyelid in relation to an ocular surface.

FIGS. 2A-2C show the change in vision seen among subjects after 2 weeks, 1 month, 1.5 months, and 3 months of treatment as measured by an Ocular Surface Disease Index (OSDI) Vision Function Sub-Scale.

### DETAILED DESCRIPTION OF THE INVENTION

Provided in a variety of embodiments herein are method of treating ocular disorders and symptoms thereof by administering a therapeutically effective amount of a pharmacologically active agent provided herein to an eye (e.g., globe or lid) of an individual in need thereof (e.g., an individual who does not wear a contact lens and/or an individual who has not been diagnosed with meibomian gland dysfunction (MGD)). In specific embodiments, the methods comprise improving vision, treating dry eye syndrome or symptoms thereof. In certain specific embodiments, the methods comprise treating dry eye syndrome or symptoms thereof. In certain embodiments, the method comprises treating dry eye, eye pain, eye inflammation, or any other disorder or symptom described herein, such as wherein such a disorder or symptom is associated vision loss or dry eye syndrome.

Provided in certain embodiments herein are methods of treating vision loss, such as by administering a keratolytic agent to the eye or its surrounding tissue (e.g., palebra conjunctiva, eyelid (e.g., eyelid margin)) of an individual in need thereof (e.g., an individual who does not wear a contact lens and/or an individual who has not been diagnosed with meibomian gland dysfunction (MGD)). In some embodiments, treatment of the vision loss comprises treating one or more symptoms associated with vision loss. In specific embodiments, a symptom associated with vision loss is a symptom identifiable by the individual and/or by a clinician. In certain embodiments, a method provided herein is associated with treating any symptom associated with vision loss, such as, by way of non-limiting example, inflammation, dryness, pain, or a combination thereof.

In specific embodiments, the keratolytic agent is selected from the group consisting of benzoyl peroxide, coal tar, dithranol, salicylic acid, selenium disulfide, alpha-hydroxy acid, urea, lactic acid, boric acid, retinoic acid, sodium thioglycolate, allantoin, zinc pyrithione, zinc L-pyrrolidone carboxylate, seleocysteine, selenomethionine, captopril, zofenopril, tiopronin, penicillamine, L-cysteine, gluthatione, dithiothreitol, thiorphan, cysteamine, bucillamine, dimercaprol, 1,1-ethanedithiol, dimercaptosuccinic acid, furan-2-ylmethanethiol, omapatrilat, ovothiol A, rentiapril, thiosalicylic acid, tixocortol, mycothiol, coenzyme A, coenzyme B, disulfiram, psammaplin A, dixanthogen, pantethine, fursultiamine, octotiamine, sulbutiamine, prosultiamine, thiram, lipoic acid, lenthionine, ajoene, allicin, gemopatrilat, thioethanol, thiophospholipid, thiocholesterol, 12-mercaptododecanoic acid, 23-(9-mercaptononyl)-3,6,9,12,15,18,21-heptaoxatricosanoic acid, and sulfanegen. In specific embodiments, the keratolytic agent is benzoyl peroxide, coal tar, dithranol, salicylic acid, selenium disulfide, alpha-hydroxy acid, urea, lactic acid, sodium thioglycolate, zinc pyrithione, or zinc L-pyrrolidone carboxylate. In some embodiments, the keratolytic agent is selenium disulfide.

Also provided in certain embodiments herein are methods of treating vision loss and/or dry eye syndrome, such as by administering a keratolytic agent to the eye or its surrounding tissue (e.g., palebra conjunctiva, eyelid (e.g., eyelid margin) or palpebra conjunctiva) of an individual in need thereof (e.g., an individual who does not wear a contact lens and/or an individual who has not been diagnosed with meibomian gland dysfunction (MGD)). In some embodiments, treatment of the dry eye syndrome and/or vision loss comprises treating symptoms associated with dry eye syndrome and/or vision loss In specific embodiments, the symptom associated with dry eye syndrome and/or vision loss is a symptom identifiable by the individual and/or by a clinician. In certain embodiments, a method provided herein is associated with treating any symptom associated with dry eye syndrome and/or vision loss such as, by way of non-limiting example, inflammation, dryness, pain, or a combination thereof. In specific embodiments, the keratolytic agent is benzoyl peroxide, coal tar, dithranol, salicylic acid, selenium disulfide, alpha-hydroxy acid, urea, lactic acid, sodium thioglycolate, zinc pyrithione, or zinc L-pyrrolidone carboxylate. In some embodiments, the keratolytic agent is selenium disulfide.

In some embodiments, the individual does not wear a contact lens.

In some embodiments, the individual has not been diagnosed with contact lens discomfort.

In some embodiments, the individual wears a contact lens.

In some embodiments, the individual has not been diagnosed with a meibomian gland dysfunction (MGD).

In some embodiments, the individual has not been diagnosed with lid wiper epitheliopathy (LWE).

In some embodiments, the individual has been diagnosed with a meibomian gland dysfunction (MGD).

In some embodiments, the individual has been diagnosed with lid wiper epitheliopathy (LWE).

In some embodiments, the individual (e.g., in need thereof) has blurry vision.

In various embodiments, therapies provided herein comprise the administration of a composition to the eye of an individual (e.g., an individual who does not wear a contact lens and/or an individual who has not been diagnosed with meibomian gland dysfunction (MGD)), or to surrounding tissue thereof (e.g., palebra conjunctiva, eyelid (e.g., eyelid margin)). Typically, for the purposes of the embodiments described herein, the eye of an individual comprises an ocular component (i.e., the eye globe or “eyeball”) and a palpebra component (e.g., comprising an upper lid and a lower lid). In certain instances, the upper and lower lids each generally comprise a palpebra conjunctiva, the palpebra conjunctiva being a tissue that lines the inside of the lid (or at least a portion thereof). In some embodiments, therapies provided herein comprise the administration of a composition to at least a portion of a palebra conjunctiva and/or eyelid margin of an individual. In some embodiments, therapies provided herein comprise the administration of a composition to a single eye of an individual. In other embodiments, therapies provided herein comprise the administration of a composition to two eyes of an individual.

In some instances, the lid-wiper region is a thickened epithelial “lip” that has a conjunctival mucosal morphology that extends from the tarsal conjunctiva up to the crest of the posterior lid border and helps to distribute the precorneal tear film.

In various embodiments discussed herein, administration of an active agent or composition described herein is achieved by administration thereof to the eye or surrounding tissue, such as the eyelid, of an individual in need thereof. In certain embodiments, topical administration to the eye of the individual comprises topical ocular administration, (e.g., topical) palpebra (lid) administration (e.g., to the inside and/or outside of the lid), or a combination thereof. In certain preferred embodiments, (e.g., direct) administration is (e.g., topical) administration to an inner lid surface. In more preferred embodiments, administration is or comprises (e.g., direct) administration to the lid wiper region of the inner lid surface. In certain embodiments, administration is or comprises (e.g., direct) administration to the lid wiper region and the stratified squamous epithelium region and/or the subtarsal fold region of the lid. In some embodiments, administration is or comprises (e.g., direct) administration to the lid wiper region, the stratified squamous epithelium region and the subtarsal fold region of the lid. In certain embodiments, administration is or comprises (e.g., direct) administration to the lid wiper, the stratified squamous epithelium, the subtarsal fold, and the stratified columnar epithelium regions of the lid. In other various embodiments, administration to the meibomian gland orifice of the lid does not also occur.

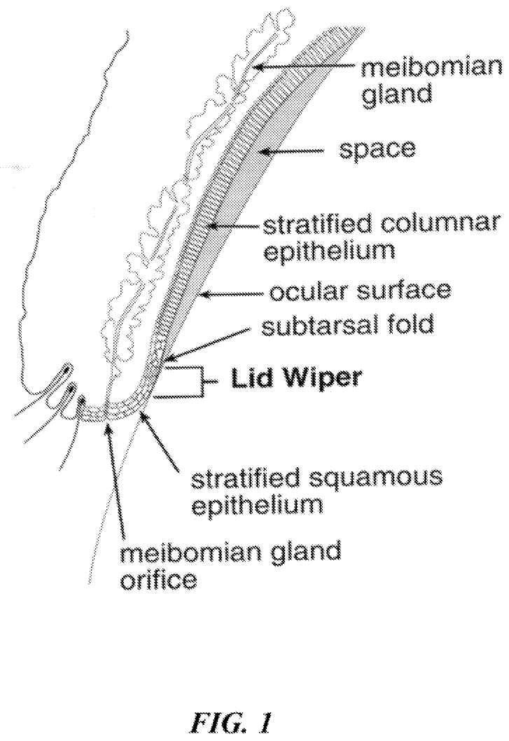

FIG. 1 illustrates a schematic of a portion of an exemplary eye surface and lid. As illustrated in the figure, at the end of the lid, eye lashes can be observed. Moving inward from the lashes, the inner surface of the lid comprises a stratified squamous epithelium region located proximal to the lashes. Further along the inner surface of the lid, the stratified squamous epithelium leads into the lid wiper region, which is the region of the inner surface that comes into contact with the ocular surface (e.g., in a normally functioning eyelid). In some instances, when an individual is suffering from a disease that affects the ocular surface resulting in vision loss, other portions of the inner surface of the lid may also come into contact with the ocular surface. Moving from the lid wiper region (e.g., moving along the inner surface of the lid in a direction distal to the lashes), the inner surface of the lid comprises a subtarsal fold region and a stratified columnar epithelium region. In some instances, a palpebra conjunctiva extends over all or a portion of the inner surface of the lid, such as having a leading edge in the lid wiper region.

The keratolytic and keratoplastic agents described herein are useful in either as an acute therapy (e.g., by a trained specialist or physician) or as a chronic therapy (e.g., in the hands of a patient or caregiver, or alternatively, by a trained specialist or physician). The agents are tested, in certain embodiments, using the assays and methods described herein (e.g., as described in the examples).

One embodiment provides a method for treating vision loss in a patient in need thereof (e.g., an individual who does not wear a contact lens and/or an individual who has not been diagnosed with meibomian gland dysfunction (MGD)) comprising topical administration of a composition comprising a keratolytic agent or keratoplastic agent. In some embodiments, the keratolytic agent is chosen from allantoin, benzoyl peroxide, inorganic selenium compounds such as selenium disulfide, SeCl₄, Na₂SeO₃, organo-selenium compounds such as Ebselen (2-phenyl-1,2-benzisoselenazol-3(2H)-one) or its analogues, coal tar, dithranol, salicylic acid, selenium disulfide, alpha-hydroxy acid, urea, lactic acid, sodium thioglycolate, zinc pyrithione, or zinc L-pyrrolodione carboxylate. In some embodiments, the keratolytic agent is selected from the group consisting of benzoyl peroxide, coal tar, dithranol, salicylic acid, selenium disulfide. In some embodiments, the keratolytic agent is selenium disulfide. In some embodiments, the keratolytic agent is salicylic acid. In some embodiments the keratolytic agent is not retinoic acid.

In certain embodiments, it is desired that the agents have minimal undesired side effects, such as causing inflammation or other adverse ocular symptoms.

In certain embodiments, a mild or weak keratolytic and/or keratoplastic agents is used in the methods and formulations described herein, e.g., with subjects that produce low levels of keratin. Such mild or weak keratolytic and/or keratoplastic agents are optionally used in a maintenance therapy setting. Mild or weak keratolytic and/or keratoplastic agents include lower concentrations of active keratolytic and/or keratoplastic agents, as well as keratolytic and/or kerotoplastic agents that have low inherent activity (as determined, e.g., by the methods described herein). In certain embodiments, the mild or weak keratolytic and/or keratoplastic agents is not boric acid.

In certain embodiments, the composition comprises a therapeutically-effective amount of at least one keratolytic agent (e.g., as described herein) in an ophthalmically-acceptable carrier. In one embodiment, the keratolytic agent is benzoyl peroxide. In another embodiment, the keratolytic agent is coal tar. In another embodiment, the keratolytic agent is dithranol. In another embodiment, the keratolytic agent is salicylic acid. In another embodiment, the keratolytic agent is selenium sulfide (e.g., selenium disulfide). As used herein, the terms “selenium sulfide” and “selenium disulfide” are used interchangeably to refer to the chemical compound having the formula SeS₂ where the ratio of selenium to sulfur is approximately 1:2. In another embodiment, the keratolytic agent is zinc pyrithione. In another embodiment, the keratolytic agent is zinc L-pyrrolidone carboxylate.

In some embodiments, more than one keratolytic agent is used.

In some embodiments, administration of a keratolytic agent to a keratin obstruction results in proteolysis of desmosomes forming tight junctions between keratinocytes. In some embodiments, administration of a keratolytic agent results in lysis, including the hydrolysis of disulfide bonds. In some embodiments, administration of a keratolytic agent reduces the production of keratin.

In some embodiments, the therapeutically effective amount of the keratolytic agent is about 0.01% or more. In some embodiments, the therapeutically effective amount of the keratolytic agent is about 0.01% to about 30% (e.g., about 0.01% to about 10%, about 0.01% to about 5%, about 0.01% to about 2.5%, about 0.01% to about 1%, about 0.1% to about 10%, about 0.1% to about 5%, about 0.1% to about 2.5%, about 0.1% to about 1%, or any range therein). In some embodiments, the therapeutically effective amount of the keratolytic agent is about 0.01% to about 10%. In some embodiments, the therapeutically effective amount of the keratolytic agent is at least about 0.01%, about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2.0%, about 2.1%, about 2.2%, about 2.3%, about 2.4%, about 2.5%, or more. In some embodiments, the therapeutically effective amount of the keratolytic agent is at least about 0.01%, such as at least about 0.05%, about 0.1%, about 1%, about 2%, about 2.5%, about 5%, about 10%, or more. In some embodiments, the therapeutically effective amount of the keratolytic agent is no more than about 10%, such as no more than about 5%, about 2.5%, about 2%, about 1%, about 0.5%, about 0.1%, or less. In some embodiments, the therapeutically effective amount of the keratolytic agent is about 0.01% to about 30%, such as about 0.01% to about 10%, about 0.1% to about 10%, or about 0.1% to about 30%.

In specific embodiments, the keratolytic agent is selenium disulfide.

In some embodiments, the composition comprises between about 0.01% to about 30% (e.g., about 0.01% to about 10%, about 0.01% to about 5%, about 0.01% to about 2.5%, about 0.01% to about 1%, about 0.1% to about 10%, about 0.1% to about 5%, about 0.1% to about 2.5%, about 0.1% to about 1%, or any useful range therein) selenium disulfide. In some embodiments, the composition comprises about 0.01% to about 10% selenium disulfide. In some embodiments, the composition comprises at least about 0.01%, about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2.0%, about 2.1%, about 2.2%, about 2.3%, about 2.4%, about 2.5%, or greater selenium disulfide. In some embodiments, the concentration of the selenium disulfide in the composition is at least about 0.01%, such as at least about 0.05%, about 0.1%, about 1%, about 2%, about 2.5%, about 5%, about 10%, or more. In some embodiments, the concentration of the selenium disulfide in the composition is no more than about 10%, such as no more than about 5%, about 2.5%, about 2%, about 1%, about 0.5%, about 0.1%, or less. In some embodiments, the composition comprises between about 0.01% to about 30% selenium disulfide, such as about 0.01% to about 10%, about 0.1% to about 10%, or about 0.1% to about 30% selenium disulfide.

In some embodiments, the composition comprising selenium disulfide is a suspension, emulsion, cream, lotion, gel (e.g., aqueous or non-aqueous), or ointment. In some embodiments, the composition comprising selenium disulfide is a semi-solid composition. In some embodiments, the composition comprising selenium disulfide is a lotion. In some embodiments, the composition comprising selenium disulfide is a cream. In some embodiments, the composition comprising selenium disulfide is an ointment. In some embodiments, the composition comprising selenium disulfide is a suspension. In some embodiments, the composition comprising selenium disulfide is a dispersion. In some embodiments, the composition comprising selenium disulfide is a solution. In other embodiments, the composition is a suspension, hydrophobic oil, foam, liposome, emulsion, lotion, microparticle, or other suitable formulation.

In some embodiments, the composition comprises inorganic selenium compounds that are inhibitors of prostaglandin synthase, the enzyme involved in the production of prostaglandins. The selenium compounds demonstrating this inhibitory effect include SeCl₄ and Na₂SeO₃. It is known that the proinflammatory action of prostaglandins enhances keratinization and therefore these water-soluble inorganic selenium compounds that interfere with the production of prostaglandin may be useful in reducing keratinization.

In some embodiments, the composition comprises organo-selenium compounds. Organo-selenium compounds such as Ebselen are antioxidant and anti-inflammatory agents inhibiting cyclooxygenase and lipooxygenase enzymes and acting as scavenger of hydrogen peroxide as well as hydroperoxides including membrane bound phospholipid and cholesterylester hydroperoxides. Anti-inflammatory agents are known to inhibit keratinization and therefore ebselen and other organo-selenium analogues may act as keratolytic agents through this antioxidant/anti-inflammatory activity.

In some embodiments, the formulation comprising the keratolytic and/or keratoplastic agent further includes an additional therapeutic agent that is not a meibomian gland opening pharmacological agent. In some embodiments the formulation does not contain jojoba wax or jojoba extract. In some embodiments the formulation does not include boric acid. In some embodiments, the formulation does not include retinoic acid. Alternatively, in some embodiments, the formulation with the keratolytic and/or keratoplastic agent excludes any additional therapeutic agent, other than an optional additional meibomian gland opening pharmacological agent.

In certain embodiments, a composition comprises (e.g., further comprises) a local anesthetic. In some embodiments, the local anesthetic chosen from an aminoamide local anesthetic, or an aminoester local anesthetic.

The term “local anesthetic” as used herein refers to an agent that induces a reversible absence of pain sensation. In some embodiments, a local anesthetic may also induce temporary muscle paralysis in addition to inducing a reversible absence of pain sensation.

The local anesthetic agents described herein are useful primarily as an acute therapy, e.g., under the guidance of a physician or other trained specialist. The agents are tested, in certain embodiments, using the assays and methods described herein.

In some embodiments, the local anesthetic is an aminoamide. In some embodiments, the local anesthetic is an aminoester. In some embodiments, the local anesthetic comprises a combination of two or more local anesthetics. In some embodiments, the combination comprises an aminoamide local anesthetic and an aminoester local anesthetic.

In some embodiments, the local anesthetic is an aminoester selected from the group consisting of: benzocaine, chloroprocaine, cocaine, cyclomethycaine, dimethocaine, larocaine, piperocaine, propoxycaine, procaine, novocaine, proparacaine, tetracaine, and amethocaine.

In some embodiments, the local anesthetic is an aminoamide selected from the group consisting of: articaine, bupivacaine, cinchocaine, dibucaine, etidocaine, levobupivacaine, lidocaine, lignocaine, mepivacaine, prilocaine, ropivacaine, and trimecaine.

In some embodiments, the local anesthetic is a combination of lidocaine and prilocaine or a combination of lidocaine and tetracaine.

In some embodiments, the local anesthetic is a naturally derived local anesthetic. In some embodiments, the naturally derived local anesthetic is selected from the group consisting of: saxitoxin, neosaxitoxin, tetrodotoxin, menthol, eugenol, and cocaine.

In some embodiments, the local anesthetic is mixed with a vasoconstrictor to increase the duration of the local anesthesia by constricting blood vessels. In some embodiments, priolocaine hydrochloride is mixed with epinephrine. In some embodiments, lidocaine, bupivacaine are mixed with epinephrine. In some embodiments, iontocaine is mixed with lidocaine and epinephrine. In some embodiments, septocaine is mixed with a combination of articaine and epinephrine. In some embodiments, local anesthetic, bupivacaine or lidocaine are mixed in combination with a steroid.

In some embodiments, the (e.g., topical) compositions described herein are combined with a pharmaceutically suitable or acceptable carrier (e.g., a pharmaceutically suitable (or acceptable) excipient, physiologically suitable (or acceptable) excipient, or physiologically suitable (or acceptable) carrier). Exemplary excipients are described, for example, in Remington: The Science and Practice of Pharmacy (Gennaro, 21^{st} Ed. Mack Pub. Co., Easton, PA (2005)). Other additives, such as a preservative is optionally provided.

In certain embodiments, compositions provided herein comprise any suitable additional agent or additive. In specific embodiments, additives are included, such as to improve performance and/or efficacy of a composition or formulation provided herein. In some instances, for example, a composition provided herein comprises a penetration enhance and/or surfactant (e.g., ionic, anionic, cationic, non-ionic, lipid (e.g., oleic or caprylic), BNZ, or the like). In some instances, a composition provided herein comprises an excipient that otherwise improves drug penetration and/or functions to solubilize plaque or keratinization, such as present on an eyelid or lens, such as according to a process described herein.

In certain specific embodiments, the pharmaceutically acceptable composition consists essentially of the at least one keratolytic agent (e.g., as described herein) and the ophthalmically-acceptable carrier. In certain specific embodiments, the pharmaceutically acceptable composition consists of the at least one keratolytic agent (e.g., as described herein) and the ophthalmically-acceptable carrier.

Described herein are methods for treating various ocular disorders (e.g., dry eye syndrome) in an individual (e.g., patient) in need comprising topical administration of a composition described herein to an individual (e.g., patient) (e.g., to the inner surface of one or more eyelid thereof) in need thereof. In some embodiments, the individual is a patient, such as a patient under medical care (e.g., for an ocular condition and/or another condition). In some instances, such therapies constitute acute therapy, such as wherein, in some embodiments, a stronger pharmacological agent (either in terms of concentration of the agent or the inherent activity of the agent) is utilized. A maintenance use, in one embodiment, allows for the use of lower concentrations of the agent, or agents with lower inherent activity. A maintenance use, in one embodiment, involves a patient at a routine visit to the health care provider. Both acute uses and maintenance uses optionally involve use of an eye-protecting device or apparatus. In one embodiment, the acute use is performed by the health care provider, and the maintenance use is performed by the patient or non-health care provider, such as a caregiver. In some embodiments, the patient applies the pharmacological agent (e.g., a composition comprising a keratolytic agent) him/herself (e.g., to the inner surface of one or more eyelid thereof). In one embodiment, such administration occurs over an extended period of time; one way of describing this patient-administered multi-administration mode is as a chronic use. In general, different or second formulations of the pharmacological agent are recommended for chronic or patient-administered uses. In one embodiment the different or second formulation utilizes a lower concentration of the pharmacological agent. In another embodiment, the second or different formulation utilizes a pharmacological agent that has a lower activity than the first formulation.

In some embodiments, the topical administration of the composition comprising a therapeutically-effective amount of at least one keratolytic agent in an ophthalmically-acceptable carrier occurs until the (e.g., abnormal) keratinization (e.g., parakeratinization (pk)) is reduced. In some embodiments, the topical administration of the composition comprising a therapeutically-effective amount of at least one keratolytic agent in an ophthalmically-acceptable carrier occurs periodically after keratinization reduction is achieved. In some embodiments, the topical administration of the composition comprising a therapeutically-effective amount of at least one keratolytic agent in an ophthalmically-acceptable carrier is a single administration. In some embodiments, the topical administration of the composition comprising a therapeutically-effective amount of at least one keratolytic agent in an ophthalmically-acceptable carrier is a periodic administration. In some embodiments, the topical administration of the composition comprising a therapeutically-effective amount of at least one keratolytic agent in an ophthalmically-acceptable carrier occurs once per day. In some embodiments, the topical administration of the composition comprising a therapeutically-effective amount of at least one keratolytic agent in an ophthalmically-acceptable carrier occurs twice per day. In some embodiments, the topical administration of the composition comprising a therapeutically-effective amount of at least one keratolytic agent in an ophthalmically-acceptable carrier occurs twice per week. In some embodiments, the topical administration of the composition comprising a therapeutically-effective amount of at least one keratolytic agent in an opthalmically-acceptable carrier occurs at night or in the evening. In some embodiments, the topical administration of the composition comprising a therapeutically-effective amount of at least one keratolytic agent in an opthalmically-acceptable carrier is followed by a period of time without exposure of the eye to light.

In some embodiments, the composition for topical administration, comprising a therapeutically-effective amount of at least one keratolytic agent in an ophthalmically-acceptable carrier is a semi-solid composition. In some embodiments, the composition for topical administration, comprising a therapeutically-effective amount of at least one keratolytic agent in an ophthalmically-acceptable carrier is homogenous. In some embodiments, the composition for topical administration, comprising a therapeutically-effective amount of at least one keratolytic agent in an ophthalmically-acceptable carrier is a dispersion. In some embodiments, the composition for topical administration, comprising a therapeutically-effective amount of at least one keratolytic agent in an ophthalmically-acceptable carrier is hydrophilic. In some embodiments, the composition for topical administration, comprising a therapeutically-effective amount of at least one keratolytic agent in an ophthalmically-acceptable carrier has an oleaginous base. In some embodiments, the composition for topical administration, comprising a therapeutically-effective amount of at least one keratolytic agent in an ophthalmically-acceptable carrier has at least one ophthalmically-acceptable excipient. In certain embodiments, the composition for topical administration is a gel, such as a non-aqueous gel.

In certain preferred embodiments, a semi-sold or other viscous formulation is utilized (e.g., a gel (e.g., gel emulsion suspension foam), cream or ointment, or other formulation, such as a suspension, hydrophobic oil, foam, liposome, emulsion, lotion, microparticle, or the like). In some instances, such formulations facilitate maintaining the pharmacologically active formulation at or near the site to be treated (e.g., the site of dysfunction, such as trauma, abnormal keratinization, or the like). In some instances, a semi-solid or other viscous formulation does little migration from the administration site.

In some embodiments, the topical administration of the composition comprising a pharmacological agent occurs once per week. In some embodiments, the topical administration of the composition comprising a pharmacological agent occurs twice per week. In some embodiments, the topical administration of the composition comprising a pharmacological agent occurs every other day. In some embodiments, the topical administration of the composition comprising a pharmacological agent occurs every day. In some embodiments, the topical administration of the composition comprising a pharmacological agent occurs several times per day.

In some embodiment, the method comprises treatment in an acute treatment scenario. In another embodiment, the method comprises treatment of a patient naïve to treatment. In another embodiment, the method comprises treatment in a chronic treatment scenario. In another embodiment, the method comprises treatment in a maintenance therapy scenario. In an acute treatment scenario, the administered dosage of pharmacological agent may be higher than the administered dosage of pharmacological agent employed in a chronic treatment scenario or a maintenance therapy scenario. In an acute treatment scenario, the pharmacological agent maybe different from the pharmacological agent employed in a chronic treatment scenario. In some embodiments, the course of therapy begins in the initial phase of therapy as an acute treatment scenario and later transitions into a chronic treatment scenario or a maintenance therapy scenario. In some embodiments, the pharmacological agent administered in the acute treatment scenario is a local anesthetic, and the pharmacological agent administered in the chronic treatment scenario or a maintenance therapy scenario is a keratolytic agent and/or keratoplastic agent. In some embodiments, the pharmacological agent administered in the acute treatment scenario is a keratolytic agent and/or keratoplastic agent, and the pharmacological agent administered in the chronic treatment scenario or a maintenance therapy scenario is a keratolytic agent and/or keratoplastic agent.

In certain clinical presentations, patients may require an initial treatment administered by a physician or healthcare professional, either by placing a more highly concentrated formulation of one of the therapeutic agents described herein. In the event the higher concentration formulations are required, the application thereof may require ocular shielding or other activity to minimize the impact of irritation or disruption of the ocular surface or surrounding tissues. Following such a procedure, a patient may be given a different formulation of active agent to take home to apply periodically to the inner surface of the lid. Such application may occur twice daily, once per day, weekly, twice per week, biweekly, or monthly, depending on the formulation activity and the desired product profile of the therapy.

In some embodiments, a method provided herein provides improvements in one or more symptoms of dry eye syndrome such as dryness, grittiness, scratchiness, soreness, irritation, burning, or watering. In some embodiments, improvements in such symptoms can be evaluated according to an Ocular Surface Disease Index (OSDI), subjective vision assessment (e.g., VAS) or another subjective scoring system (e.g., CLDEQ-8). In some embodiments, a method provided herein provides improvements in eyelid signs associated with vision loss and/or dry eye syndrome. In some embodiments, a method provided herein provides improvements in the tear film signs associated with vision loss and/or dry eye syndrome. In some embodiments, a method provides improvements in the CLDEQ-8 measurement tool. In some embodiments, a method provides improvements in vision. In some embodiments, a method provides improvements in subjective vision assessment (e.g., as assessed by an ocular surface disease index (OSDI) or visual analogue scale, VAS). In some embodiments, an improvement in one or more symptoms of vision loss and/or dry eye syndrome, is observed within about one month of administration of a composition (e.g., according to a method provided herein), such as within about three months, two months, six weeks, four weeks, three weeks, two weeks, one week, three days, two days, or sooner. In some embodiments, an improvement in one or more symptoms of vision loss and/or dry eye syndrome, is observed within about three months of administration of a composition (e.g., according to a method provided herein). In some embodiments, improvement in a symptom of vision loss and/or dry eye syndrome continues over a period of administration, such as over one week, two weeks, three weeks, four weeks, one month, two months, three months or longer.

In some embodiments, a method provided herein further comprises one or more additional therapeutic interventions such as application of a warm compress, debridement, and/or therapeutic expression (e.g., manual expression or physical expression using an instrument such as LipiFlow). In some embodiments, a method provided herein further comprises performing debridement or debridement coupled with therapeutic expression in an eye of an individual. In some embodiments, debridement is performed in one eye and debridement coupled with therapeutic expression is performed in another eye. In some embodiments, a therapeutic intervention (also referred to herein as a physical intervention) is performed after administration of a composition provided herein. For example, in some embodiments, the physical intervention is performed at least about 5 minutes after administration of the composition, such as at least about 10 minutes, about 20 minutes, about 30 minutes, about 1 hour, about 2, hours, about 4 hours, about 8 hours, about 12 hours, about 24 hours, about 48 hours, about 72 hours, about 1 week, about 2 weeks, about 3 weeks, about 4 weeks, about 1 month, six weeks, three months or longer after administration of a composition provided herein. In some embodiments, the composition is administered periodically and the physical intervention is performed a single time, such as about 1 month after an initial administration of the composition.

One aspect of the methods of treatment described herein is the location of the topical administration of the composition. In one embodiment, the composition comprising a pharmacological agent is administered such that little or no irritation to eye or its surrounding tissue occurs. In one embodiment, the composition comprising a pharmacological agent (e.g., keratolytic agent) is administered to an inner surface of an eyelid of an individual in need thereof (e.g., one or both upper lid and/or one or both lower lid).

One additional embodiment of the methods of treatment described herein is the use of a protective element provided to the eye to avoid irritation to the eye. Although the formulations described herein are generally non-irritating, in some embodiments (e.g., high concentration of agent or when used on a sensitive eye) a protective element provides an additional layer of safety and comfort for the patient. In one embodiment, the composition comprising a pharmacological agent is administered while an eye shield is placed on the eye to reduce contact of the pharmacological agent with the cornea and/or conjunctiva such that reduced irritation to eye occurs. In some embodiments, the eye shield is a contact lens or an eye covering. In some embodiments, the eye covering comprises a self-adhesive. In one embodiment, the composition comprising a pharmacological agent is administered while the lid is pulled away from the globe to reduce contact of the pharmacological agent with the cornea and/or conjunctiva such that reduced irritation to eye occurs.

As used herein and in the appended claims, the singular forms “a,” “and,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range may vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, may “consist of” or “consist essentially of” the described features.

The terms “treat,” “treating,” or “treatment” as used herein, include reducing, alleviating, abating, ameliorating, relieving, or lessening the symptoms of a disorder described herein, such as vision loss and/, in either a chronic or acute therapeutic scenario. In one embodiment, treatment includes a reduction of a terminal duct obstruction.

The term “recurrence,” or “reducing relapse” symptoms of a disorder described herein, such as vision loss and/, in a chronic therapeutic scenario.

The term “keratolytic agent” and/or “keratoplastic agent” as used herein refers to an agent that softens, disrupts, dissolves, solubilizes, or loosens a keratinized obstruction, or prevents the formation of a keratinized obstruction. Specifically, the term “Keratolytic agents” refers to agents used to promote softening and dissolution of keratin and the term “keratoplastic agents” refers to agents used to reduce keratin production.

The term “lotion” describes an emulsion liquid dosage form. This dosage form is generally for external application to the skin (US FDA Drug Nomenclature Monograph, number C-DRG-00201).

The term “cream” describes an emulsion semisolid dosage form, usually containing >20% water and volatiles and/or <50% hydrocarbons, waxes or polyols as the vehicle. A cream is more viscous than a lotion. This dosage form is generally for external application to the skin (US FDA Drug Nomenclature Monograph, number C-DRG-00201).

The term “ointment” describes a semisolid dosage form, usually containing <20% water and volatiles and/or >50% hydrocarbons, waxes or polyols as the vehicle. This dosage form is generally for external application to the skin or mucous membranes (US FDA Drug Nomenclature Monograph, number C-DRG-00201).

The term “solution” describes a clear, homogeneous liquid dosage form that contains one or more chemical substances dissolved in a solvent or mixture of mutually miscible solvents (US FDA Drug Nomenclature Monograph, number C-DRG-00201).

The term “suspension” refers to a heterogeneous mixture containing solid particles that are not dissolved, but can get suspended throughout at least a portion of the bulk of the solvent.

Concentrations of agents provided herein are based on any suitable measurement, such as wt. %, w/w %, or w/v %. In specific instances, the concentration is wt. % (e.g., w/w % or w/v %).

The term “about” means any acceptable amount, such as suitable to achieve the stated purpose. In some instances, “about” refers to, for example, plus or minus 20%, or plus or minus 10%, or plus or minus 5%.

The term “comprising,” as used herein, also includes an explicit disclosure of “consisting of” and “consisting essentially of.”

### EXAMPLES

### Example 1: Pharmacological Active Formulations

High viscosity formulations are prepared, such as for administration according to the disclosures provided herein. Any suitable formulation, such as a cream, ointment, emulsion, suspension, microspheres, or the like are optionally utilized. In various embodiments, exemplary ophthalmic ointment formulations are prepared according to the following formulations:

| Ingredient | % weight/volume |
|---|---|
| Water | 3% |
| 80% White Petrolatum | 90% |
| 20% Mineral Oil | 3% |
| Liquid Lanolin | 3% |
| Salicylic acid | 1% |

| Ingredient | % weight/volume |
|---|---|
| Water | 3% |
| 80% White Petrolatum | 90.9% |
| 20% Mineral Oil | 3% |
| Liquid Lanolin | 3% |
| Salicylic acid | 0.1% |

| Ingredient | % weight/volume |
|---|---|
| Water | 3% |
| 80% White Petrolatum | 90% |
| 20% Mineral Oil | 3% |
| Liquid Lanolin | 3% |
| Selenium disulfide | 1% |

| Ingredient | % weight/volume |
|---|---|
| Water | 3% |
| 80% White Petrolatum | 90.9% |
| 20% Mineral Oil | 3% |
| Liquid Lanolin | 3% |
| Selenium disulfide | 0.1% |

Other formulations, such as ointment/semi-solid, surfactant formulations are contemplated and provided herein.

Abnormal keratitis is induced in the eyes of rabbits using a 0.5% benzalkonium chloride solution. Each rabbit is assigned to receive an ointment provided herein in one eye and a control ointment in the other eye. Evaluation of the inner surface of the lid, such as in the lid wiper and/or conjunctival folds is evaluated.

### Example 2: Clinical Evaluation of Vision Improvement

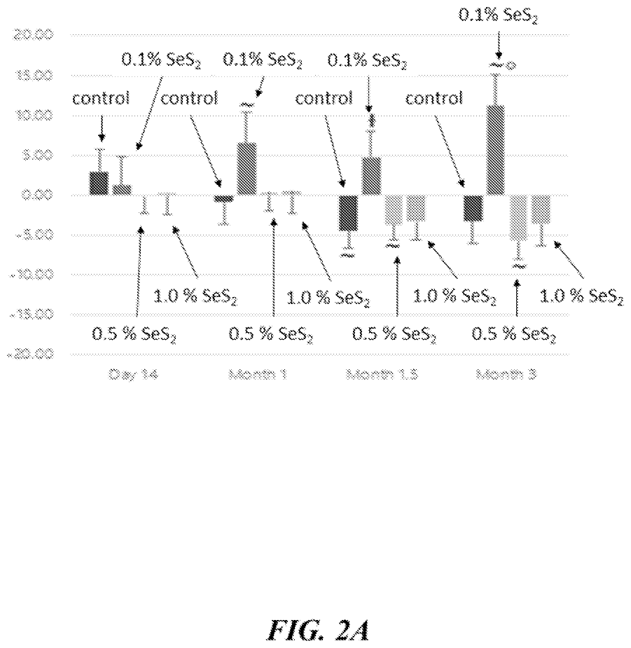

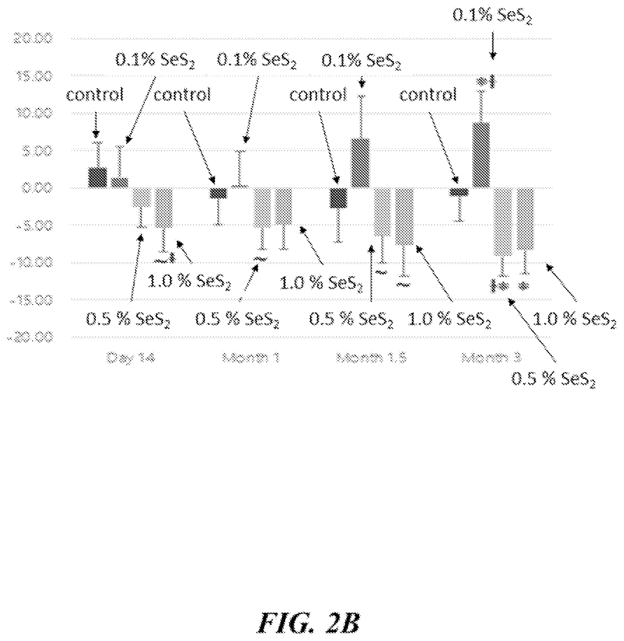

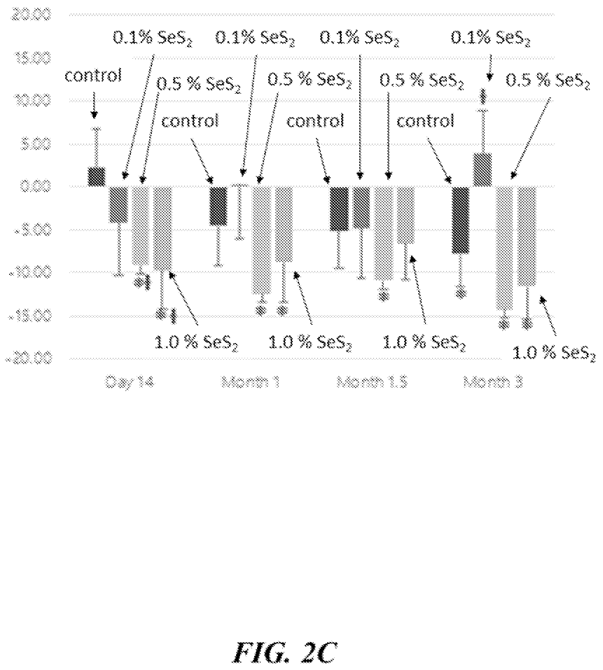

Patients with vision loss were treated with control (N=26) or a drug formulation comprising 0.1% selenium disulfide (n=9), 0.5% selenium disulfide (n=26), or 1% selenium disulfide (n=34). Treatment consisted of twice a week application of the drug product on the lower eyelid of both eyes. Patients were followed up and their vision was assessed with an Ocular Surface Disease Index (OSDI) vision assessment after two weeks, one month, six weeks, and 3 months. FIGS. 2A-2C shows patients change from baseline as assessed by the OSDI vision assessment (e.g., based on: OSDI ocular symptoms sub-scale, change from baseline (FIG. 2A), OSDI vision function sub-scale, change from baseline (FIG. 2B), and OSDI environmental triggers sub-scale, change from baseline (FIG. 2C)). As can be seen in FIGS. 2A-2C, not only did patients treated with 0.5% selenium disulfide and 1% selenium disulfide have an improvement in vision, patients also showed continued improvement from 2 weeks to three months. † represents a significant difference from control (p<0.10); ° represents a significant difference from control (p<0.05); ˜ represents a significant difference from baseline (p<0.10); * represents a significant difference from baseline (p<0.05).

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A method of improving vision in an individual in need thereof, the method comprising providing to the individual a composition comprising a keratolytic agent in an ophthalmically or pharmaceutically acceptable vehicle or carrier, the composition being administered to the eyelid or the eye of the individual in a manner suitable to deliver the keratolytic agent to an eyelid margin of the eyelid or ocular surface of the individual.

2. The method of claim 1, wherein the composition is provided to the individual periodically.

3. The method of any one of the preceding claims, wherein the composition is periodically administered to the individual over a time period of at least 2 weeks.

4. The method of any one of the preceding claims, wherein the composition is periodically administered to the individual over a time period of at least 1 month.

5. The method of any one of the preceding claims, wherein periodic administration comprises administering the composition at least once a week.

6. The method of any one of the preceding claims, wherein periodic administration comprises administering the composition at least twice a week.

7. The method of claims 1-6, wherein the individual has not been diagnosed with a meibomian gland dysfunction (MGD) or lid wiper epitheliopathy (LWE).

8. The method of claims 1-6, wherein the individual has been diagnosed with a meibomian gland dysfunction (MGD) or lid wiper epitheliopathy.

9. The method of claims 1-8, wherein the individual does not wear a contact lens.

10. The method of claim 1-8, wherein the individual does wear a contact lens.

11. The method of any one of the preceding claims, wherein a concentration of the keratolytic agent in said composition is between about 0.01% to about 10% by weight.

12. The method of any one of the preceding claims, wherein said keratolytic agent is selenium disulfide.

13. The method of claim 12, wherein said composition comprises 0.1% by weight selenium disulfide in an ophthalmically acceptable carrier.

14. The method of claim 12, wherein said ophthalmically acceptable carrier comprises at least one ophthalmically acceptable vehicle and at least one ophthalmically acceptable excipient.

15. The method of any one of the preceding claims, wherein the composition is administered to at least a portion of a palebra conjunctiva of said eye of said individual.

16. The method of any one of the preceding claims, wherein said composition is a dispersion or suspension.

17. The method of any one of the preceding claims, wherein said composition is hydrophilic.

18. The method of any one of the preceding claims, wherein said composition comprises an oleaginous base.

19. The method of any one of the preceding claims, wherein said composition is homogeneous.

20. The method of any one of the preceding claims, wherein said composition is administered via an applicator.

21. The method of claim 20, wherein said applicator is a finger of said individual.

22. The method of any one of the preceding claims, wherein the vision is improved by either questioning patients of their visual function, or improvement measured using a subjective measure or improvement measured using an objective measure.

23. The method of any of the preceding claims, wherein the questioning patients of their visual function uses a questionnaire.

24. The method of any one of the preceding claims, where the questionnaire can be OSDI, VAS, or CLDEQ-8.

25. The method of any one of the preceding claims, wherein said vision function is improved by at least two points as measured by an OSDI vision function scale after one month of periodic administration.

26. The method of any one of the preceding claims, wherein said vision function is improved by at least five points as measured by an OSDI vision function scale after two months of periodic administration.

27. The method of any one of the preceding claims, wherein said vision function is improved in aspects including reading, driving at night, working with a computer and watching TV

28. The method of any one of the preceding claims, wherein said subjective measure is any single or combination of visual acuity, hyperacuity, contrast sensitivity, glare visual acuity, visual acuity using PAM, visual acuity under photopic conditions and visual acuity under mezopic conditions.

29. The method of any one of the preceding claims, wherein said objective measure is any single or combination of a Visual Quality Aberrometer, Objective Scatter Image, and a Double Pass System.
